(19) United States
(12) Patent Application Publication
(10) Pub. No.: US 2009/0270325 A1
(43) Pub. Date: Oct. 29, 2009

(54) GROWTH FACTOR

(75) Inventors: Richard Ross, Sheffield (GB); Peter Artymiuk, Sheffield (GB); Jon Sayers, Sheffield (GB)

Correspondence Address: MORGAN, LEWIS & BOCKIUS LLP (SF), One Market, Spear Street Tower, Suite 2800, San Francisco, CA, 94105, US

(73) Assignee: ASTERION LIMITED, Sheffield (GB)

(21) Appl. No.: 12/441,361

(22) PCT Filed: Sep. 13, 2007
(86) PCT No.: PCT/GB2007/003453

§ 371 Date: Mar. 19, 2009

(30) Foreign Application Priority Data
Sep. 14, 2006 (GB) 0618082.2

(51) Int. Cl. A61K 38/16; C12N 15/11; C07K 14/00; C12N 15/00; C12N 5/06

(52) U.S. Cl. 514/12; 536/23.4; 530/350; 435/320.1; 435/358

(57) ABSTRACT

We describe a chimeric protein comprising a growth hormone polypeptide linked to a polypeptide comprising the extracellular binding domain of growth hormone receptor; its use in enhancing the growth and metabolism of non-human animals and homodimers comprising said chimeric protein.

The invention relates to a chimeric polypeptide comprising a growth hormone polypeptide linked to a polypeptide comprising the extracellular binding domain of growth hormone receptor and its use in enhancing the growth and metabolism of non-human animals, typically livestock animals.

Growth hormone, also known as somatotropin, is a protein hormone of about 190 amino acids and is synthesized and secreted by the cells of the anterior pituitary. It functions to control several complex biological processes including growth and metabolism. Growth hormone can have direct effects through binding growth hormone receptor expressed by responsive cells and indirect effects which are primarily mediated by insulin-like growth, factor (IGF-I), a hormone secreted by the liver and other tissues in response to growth hormone. A major role of growth hormone is therefore the stimulation of the liver to produce IGF-I. IGF-I stimulates, amongst other cells, the proliferation of chondrocytes resulting in bone growth. IGF-I is also implicated in muscle development.

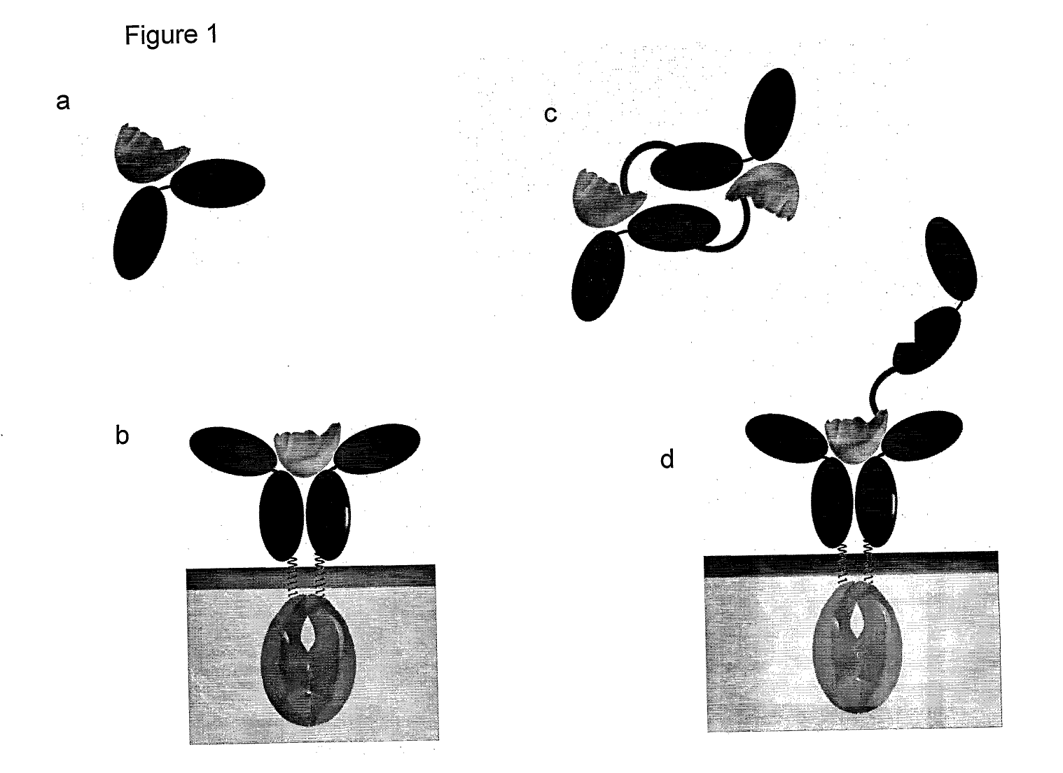

GH acts through a cell surface receptor (GHR) which is a member of the type 1 cytokine receptor family. Cytokine receptors have a single transmembrane domain and dimerization or oligomerisation is required to activate intracellular signalling pathways. In common with other cytokine receptors the extracellular domain of the GHR is proteolytically cleaved and circulates as a binding protein (FIG. 1). Under physiological conditions GH is in part bound in the circulation and the complex with the binding protein is presumed to be biologically inactive and protected from clearance and degradation. Co-administration of binding protein with GH in vivo delays GH clearance and augments its anabolic action. Thus, like many hormonal systems binding in the circulation provides an inactive circulating reservoir in equilibrium with active free hormone.

In our co-pending application WO01/96565 we disclose cytokine agonists useful in the treatment of diseases and conditions that result from, for example growth hormone deficiency. We herein disclose a chimeric molecule comprising growth hormone optionally linked via a linker molecule to an extracellular domain of growth hormone receptor. We have conducted animal experiments using rats deficient in pituitary function which are consequently deficient in growth hormone. Surprisingly, chimeric molecules have been found to have greater activity than native growth hormone in growth hormone replacement therapy and have a much extended half life when compared to other growth hormone chimeras and growth hormone. This may be related to a property of the chimeric molecules to form homodimers with each other. This is unexpected since in vitro bioassays indicate that the chimeric molecules disclosed in WO01/96565 have a lower affinity for growth hormone receptor and consequently low activity in cell based assays when compared to native growth hormone. Moreover, chimeric molecules disclosed in WO01/96565 also enhance the secretion of insulin-like growth factor when compared to native growth factor. We disclose the in vivo activity of growth hormone super agonists and their use in promoting animal growth and metabolism, in particular in boosting meat and milk production in livestock.

According to an aspect of the invention there is provided the use of a chimeric growth hormone agonist which is a fusion protein comprising: a first part comprising growth hormone, or a receptor binding domain thereof, optionally linked by a peptide linking molecule to a second part comprising the extracellular binding domain of growth hormone receptor for the enhancement of the growth and/or metabolism of a non-human animal species.

According to a further aspect of the invention there is provided the use of a chimeric growth hormone agonist which is a fusion protein comprising: a first part comprising growth hormone, or a receptor binding domain thereof, optionally linked by a peptide linking molecule to a second part comprising the extracellular binding domain of growth hormone receptor for the manufacture of a composition for the enhancement of the growth and/or metabolism of a non-human animal species.

In a preferred embodiment of the invention said peptide linking molecule consists of 5-30 amino acid residues.

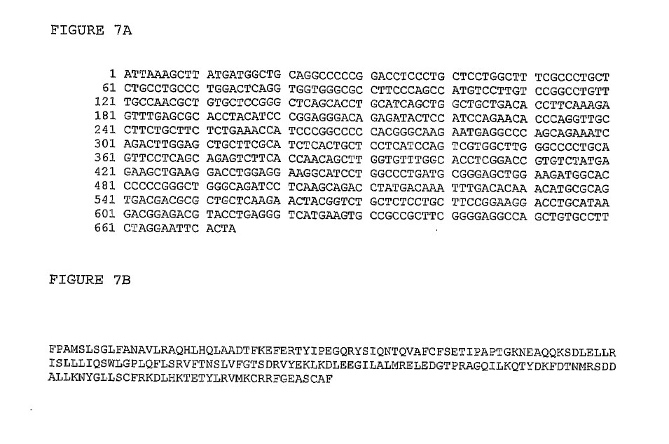

In a preferred embodiment of the invention said fusion protein comprises a polypeptide encoded by a nucleic acid molecule as represented by the nucleic acid sequence in FIG. 7a, or a variant nucleic acid molecule that hybridizes under stringent hybridization conditions to a nucleic acid molecule comprising a nucleic sequence in FIG. 7a and encodes a protein that has growth hormone activity.

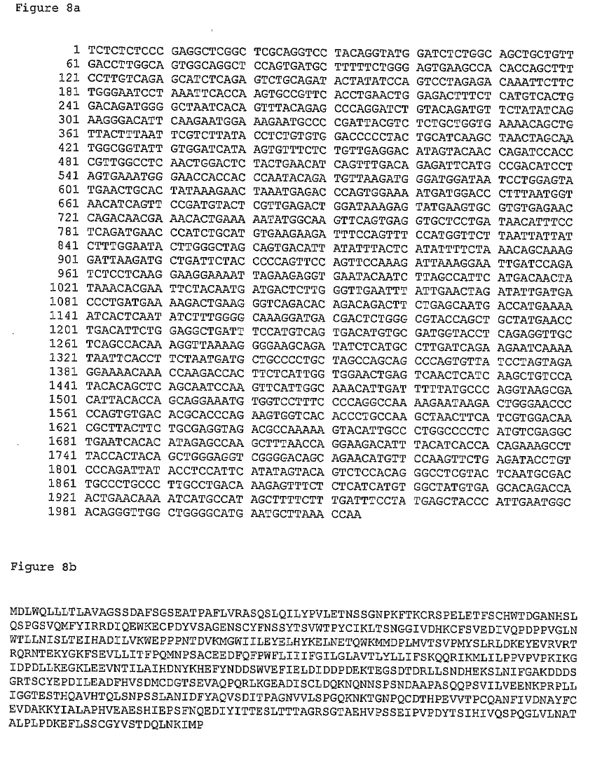

In a further preferred embodiment of the invention said fusion protein comprises a polypeptide encoded by a nucleic acid molecule as represented by the nucleic acid sequence in FIG. 8a or a variant nucleic acid molecule that hybridizes under stringent hybridization conditions to a nucleic acid molecule comprising a nucleic sequence in FIG. 8a and encodes a protein that has growth hormone receptor activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a preferred embodiment of the invention said chimeric fusion protein comprises an amino acid sequence as represented in FIG. 7b, or a variant amino acid sequence that varies from the sequence represented in FIG. 7b by addition, deletion or substitution of at least one amino acid residue and has the specific activity associated with growth hormone.

In a preferred embodiment of the invention said chimeric fusion protein comprises an amino acid sequence as represented in FIG. 8b, or a variant amino acid sequence that varies from the sequence represented in FIG. 8b by addition, deletion or substitution of at least one amino acid residue and has the specific activity associated with growth hormone receptor.

A variant polypeptide may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan.

Most highly preferred are variants that retain the same biological function and activity as the reference polypeptide from which it varies and represents species variants of the polypeptide, for example sheep, pig, horse, deer, boar, fowl, for example chicken, fish, for example salmon or goat growth hormone or growth hormone receptor or growth hormone receptor extracellular domain.

The invention features polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein.

In a preferred embodiment of the invention said peptide linking molecule comprises at least one copy of the peptide Gly Gly Gly Gly Ser.

In a preferred embodiment of the invention there is provided a chimeric protein comprising 2, 3, 4, 5 or 6 copies of the peptide Gly Gly Gly Gly Ser.

In a preferred embodiment of the invention said chimeric protein consists of a first part consisting of growth hormone linked by a peptide which consists of 5 to 30 amino acid residues to a second part consisting of the extracellular binding domain of growth hormone receptor.

In a preferred embodiment of the invention said peptide linker consists of 5, 10, 15, 20, or 30 amino acid residues.

In an alternative embodiment of the invention said chimeric polypeptide does not comprise a peptide linking molecule and is a direct in frame translational fusion of first and second parts.

In a preferred embodiment of the invention said growth enhancement is the promotion of muscle and bone development by said animal.

In a further preferred embodiment of the invention said metabolic enhancement is the promotion of milk production by said animal.

In a further preferred embodiment of the invention said chimeric fusion protein enhances the production of insulin-like growth factor by said animal; preferably said fusion protein enhances the production of insulin-like growth factor by at least 2-fold when compared to native growth hormone; preferably said fusion protein enhances the production of insulin-like growth factor by at least 4-fold when compared to native growth hormone.

According to a further aspect of the invention there is provided a method to enhance the growth and/or metabolism of a non-human animal species comprising administering to said animal an effective amount of a chimeric fusion protein wherein said fusion protein comprises a first part comprising growth hormone, or a receptor binding domain thereof, optionally linked by a peptide linking molecule to a second part comprising the extracellular binding domain of growth hormone receptor.

In a preferred method of the invention said chimeric fusion protein comprises an amino acid sequence as represented in FIG. 7b, or a variant amino acid sequence that varies from the sequence represented in FIG. 7b by addition, deletion or substitution of at least one amino acid residue and has the specific activity associated with growth hormone.

In a preferred method of the invention said chimeric fusion protein comprises an amino acid sequence as represented in FIG. 8b, or a variant amino acid sequence that varies from the sequence represented in FIG. 8b by addition, deletion or substitution of at least one amino acid residue and has the specific activity associated with growth hormone receptor.

In a preferred method of the invention said chimeric fusion protein is administered intravenously.

In an alternative preferred method of the invention said chimeric fusion protein is administered subcutaneously.

In a further preferred method of the invention said chimeric fusion protein is administered at two day intervals to said animal; preferably said fusion protein is administered at weekly, 2 weekly or monthly intervals to said animal.

In a preferred method of the invention said animal is selected from the group consisting of: cattle, sheep, pig, horse, deer, boar, and fowl, for example chicken, fish, for example salmon; preferably said animal is a cow.

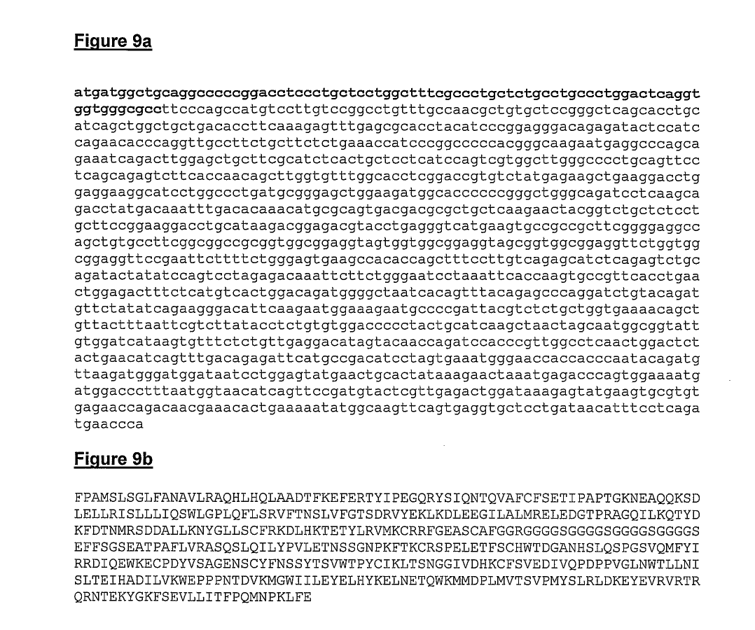

According to a further aspect of the invention there is provided a nucleic acid molecule that encodes a polypeptide as represented by the amino acid sequence in FIG. 9b.

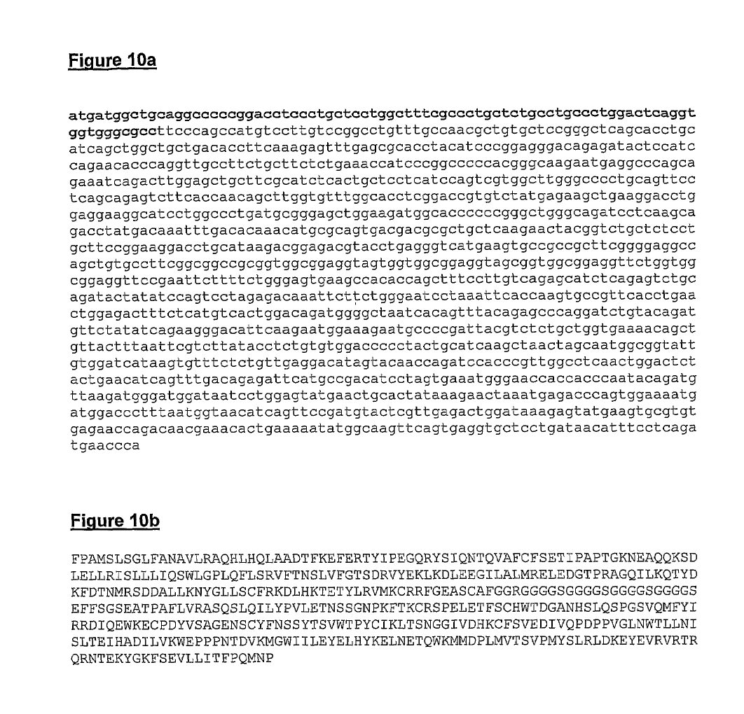

According to a further aspect of the invention there is provided a nucleic acid molecule that encodes a polypeptide as represented by the amino acid sequence in FIG. 10b.

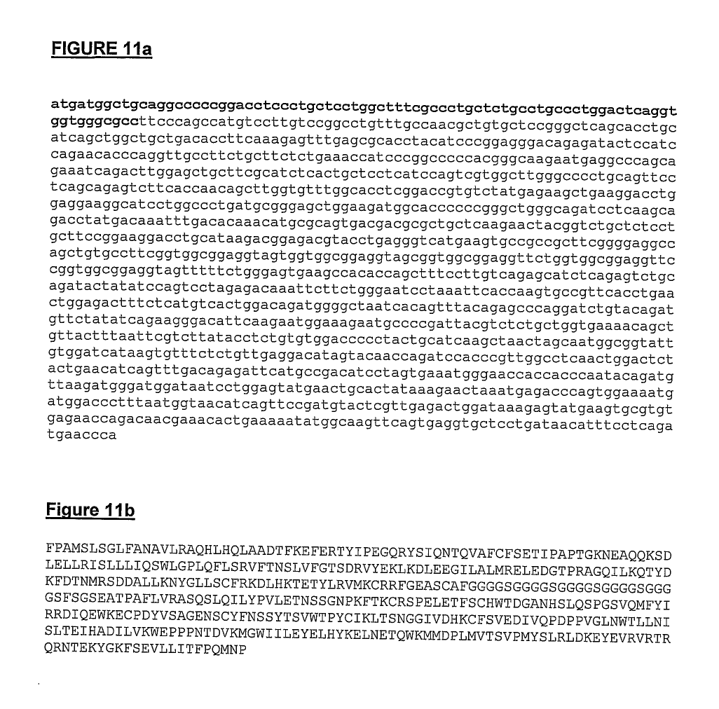

According to a further aspect of the invention there is provided a nucleic acid molecule that encodes a polypeptide as represented by the amino acid sequence in FIG. 11b.

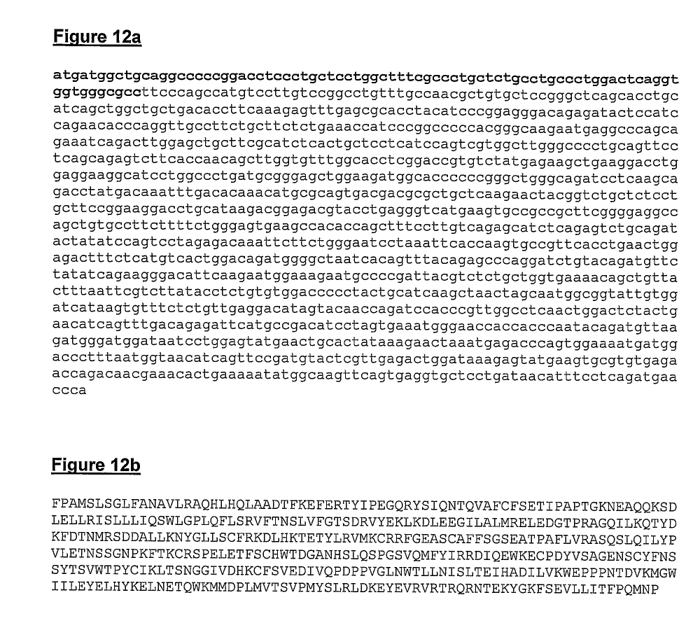

According to a further aspect of the invention there is provided a nucleic acid molecule that encodes a polypeptide as represented by the amino acid sequence in FIG. 12b.

According to an aspect of the invention there is provided a polypeptide comprising an amino acid sequence as represented in FIG. 9b.

According to an aspect of the invention there is provided a polypeptide comprising an amino acid sequence as represented in FIG. 10b.

According to an aspect of the invention there is provided a polypeptide comprising an amino acid sequence as represented in FIG. 11b.

According to an aspect of the invention there is provided a polypeptide comprising an amino acid sequence as represented in FIG. 12b.

According to an aspect of the invention there is provided an expression vector comprising a nucleic acid molecule according to the invention.

According to an aspect of the invention there is provided a cell transfected with a vector according to the invention.

According to an aspect of the invention there is provided an antibody that specifically binds a polypeptide according to the invention.

In a preferred embodiment of the invention said antibody is a monoclonal antibody or active binding part thereof.

According to a further aspect of the invention there is provided a homodimer comprising first and second polypeptides wherein said first and second polypeptides comprise: a first part comprising growth hormone, or a receptor binding domain thereof, optionally linked by a peptide linking molecule to a second part comprising the extracellular binding domain of growth hormone receptor.

In a preferred embodiment of the invention said growth hormone and the extracellular binding domain of growth hormone receptor are bovine.

In a preferred embodiment of the invention said homodimer comprises a polypeptide comprising or consisting of an amino acid sequence as represented in FIGS. 9b, 10b, 11b or 12b.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1: Schematic of relationship between GH, chimera and the GHR: (a) Shows GH bound to GHBP which is the proteolysed extracellular A & B domains of the GHR; (b) Shows GH binding to a GHR dimer which is shown with both the extracellular domain (green) and intracellular domain (blue); (c) Shows the chimera formed as a dimer in solution; and (d) shows the presumed conformation of the chimera binding and activating the GHR.

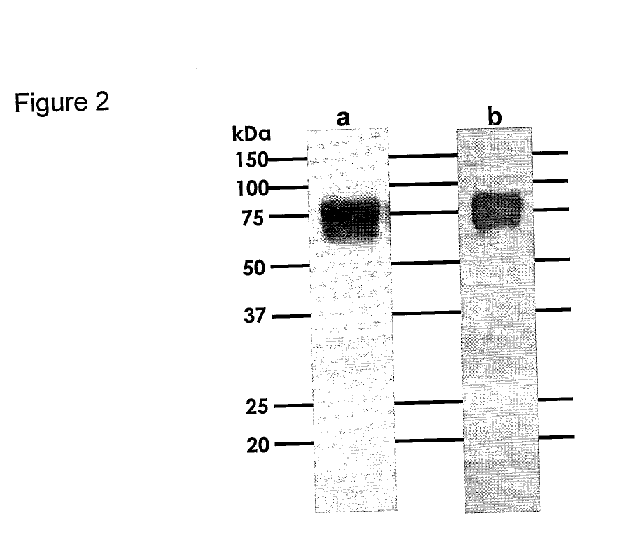

FIG. 2: Purified Chimera: (a) Coomassie gel; and (b) western blot.

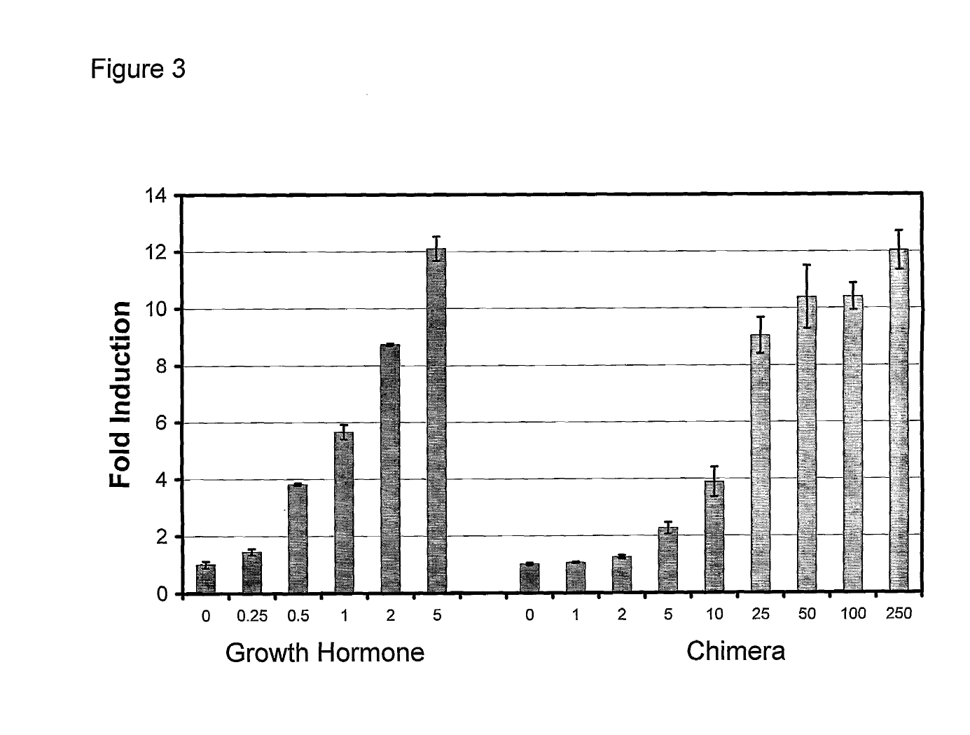

FIG. 3: Shows the results of the Bioassay. The y axis is fold-induction of corrected luciferase from a Stat 5 luciferase-reporter assay. The maximal response for GH is achieved with 5 nM concentration of GH whereas the maximal response with the chimera requires 50 to 250 nM.

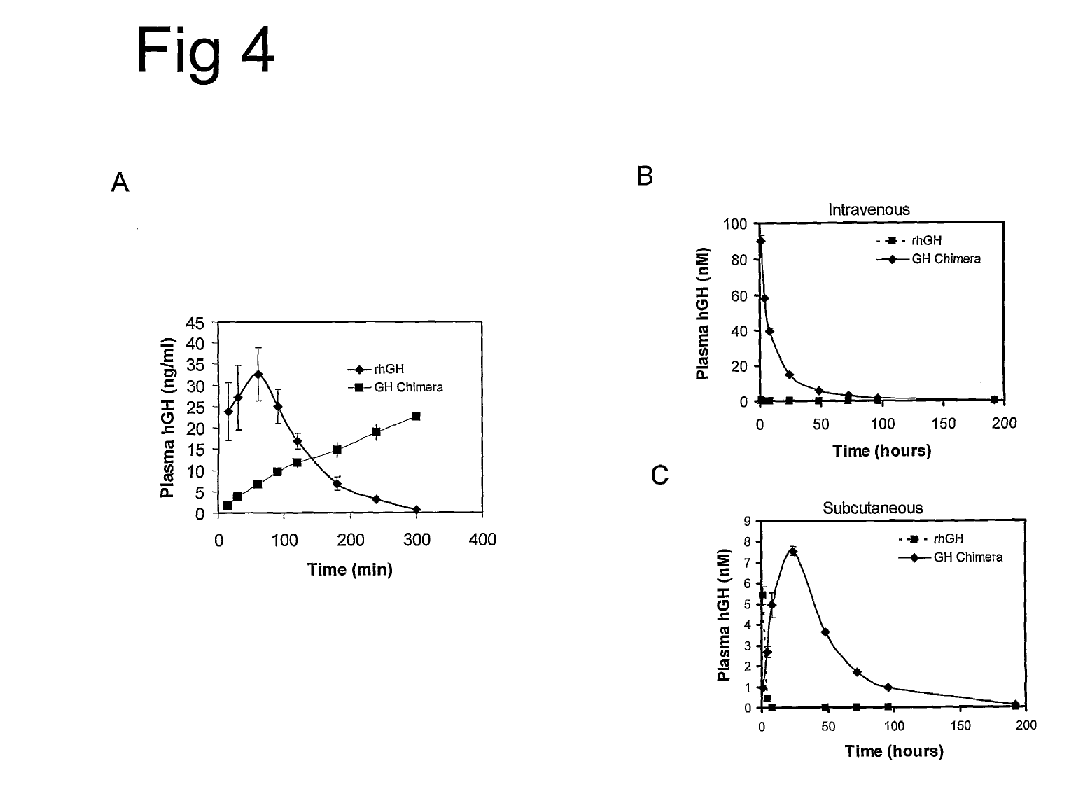

FIG. 4: Shows profiles of GH and Chimera measured after subcutaneous (sc) and intravenous (iv) administration: (a) Shows early phase after sc administration; (b) Shows late phase after iv; and (c) late phase after sc administration.

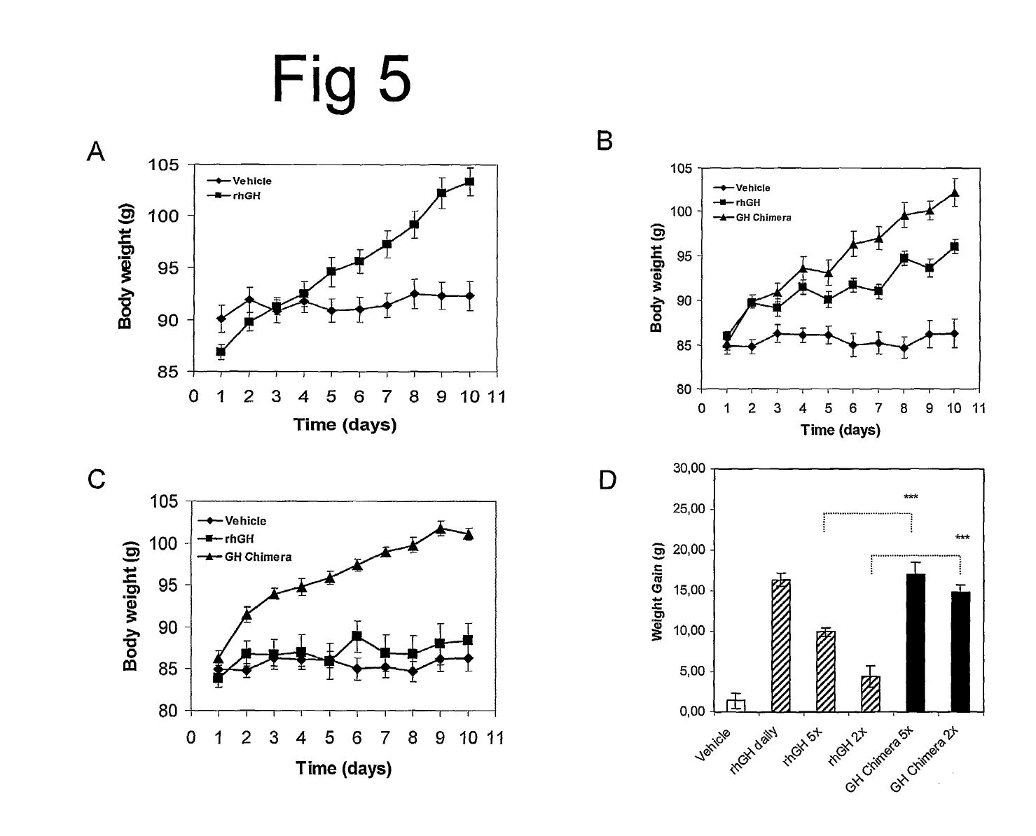

FIG. 5: Shows the body weight change after subcutaneous treatment with GH and Chimera: (a) after daily GH versus placebo (vehicle only); (b) alternate day injections; (c) two injections on days 1 and 5; and (d) summary of changes in body weight after different treatment regimens.

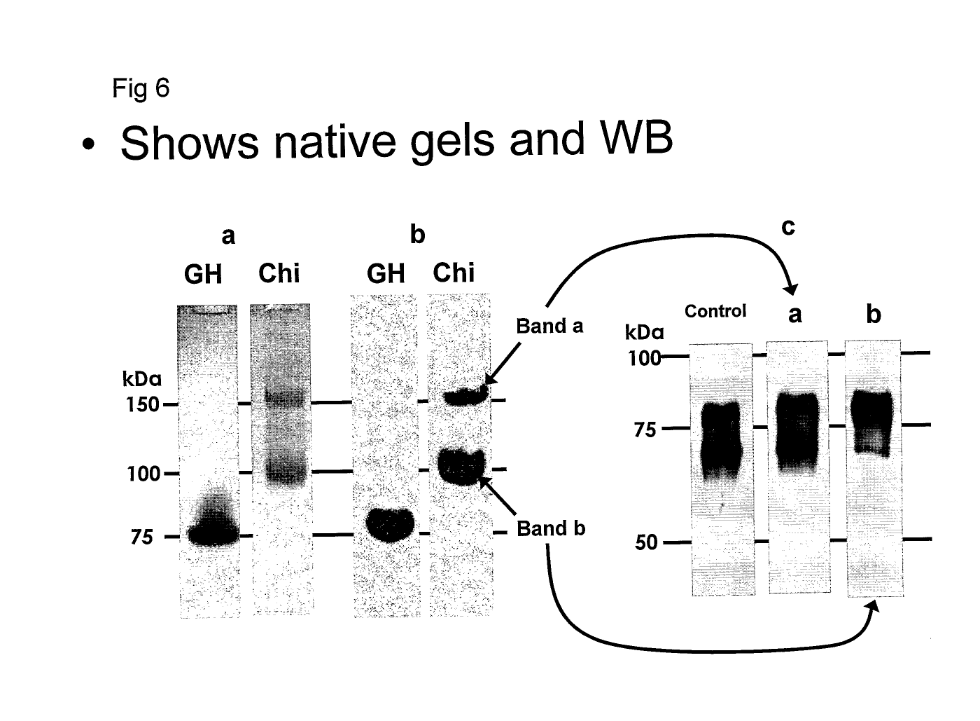

FIG. 6: Shows GH and chimera: (a) run on a native Coomassie gel and (b) western blot of a native gel. It should be noted that the size markers are not accurate on a native gel. In the native gel GH runs at an apparent MW greater than predicted and the chimera runs as two distinct bands; the higher band being approximately twice the MW of the lower band. In (c) the two bands for the chimera from the native gel have been excised, denatured and then western blotted (arrows show bands from native gel run on denatured gel. The control is the denatured 75 kDa chimera). It can been seen that both the bands from the native gel run at the predicted size of the Chimera suggesting that on the native gel we maybe seeing a monomer and dimer.

FIG. 7a is the nucleic acid sequence of bovine growth hormone; FIG. 7b is the amino acid sequence of bovine growth hormone;

FIG. 8a is the nucleic acid sequence of the extracellular domain of bovine growth hormone receptor; FIG. 8b is the amino acid sequence of the extracellular domain of bovine growth hormone receptor.

FIG. 9a illustrates the structure and DNA sequence of the bovine GH-GHR chimera IB7 (Bov) v3 (bold encodes signal sequence); FIG. 9b represents the amino acid sequence of the bovine GH-GHR chimera IB7 (Bov) v3;

FIG. 10a illustrates the structure and DNA sequence of bovine GH-GHR chimera including linker sequence 1B7 (Bov) v0 (bold encodes signal sequence); FIG. 10b represents the amino acid sequence of the bovine GH-GHR chimera with peptide linker 1B7 (Bov) v0; FIG. 10c is an alignment of bovine and human chimeras.

FIG. 11a illustrates the structure and DNA sequence of bovine GH-GHR chimera including linker sequence 1B7 (Bov)v1 (bold encodes signal sequence); FIG. 11b represents the amino acid sequence of the bovine GH-GHR chimera with peptide linker 1B7(Bov) v1;

FIG. 12a illustrates the structure and DNA sequence of bovine GH-GHR chimera including linker sequence 1B7 (Bov) v2 (bold encodes signal sequence); FIG. 12b represents the amino acid sequence of the bovine GH-GHR chimera with peptide linker 1B7 (Bov) v2.

### MATERIALS AND METHODS

Use of animals and human samples: The use of human samples was approved by the local ethics committee and patients gave informed consent. All the experiments have been conducted in compliance with the French laws (Council Directive N° 86/609/EEC of 24 Nov. 1986) relating to the protection of animals used for experimental or other scientific purpose.

Materials: All the materials were purchased from Sigma (Poole, UK) unless otherwise stated. Recombinant human GH was purchased from Pfizer, recombinant E. coli derived human GHBP used in binding assays was a gift from DSL (DSL Research Reagents, Oxfordshire, UK), and iodinated GH a gift from NovoNordisk (NovoNordisk Park, Denmark). Anti-GH and GH receptor mAbs used for purification and characterisation were in-house materials (CS) except mAbs B07b and B24a which were a gift from Dr. Skriver (NovoNordisk Park, Denmark).

### Cloning, Expression and Purification of 1B7Stop(Bovine)

### Cloning

Synthesis of the 1B7Stop (Bovine) gene required the replacement of the human GH and GHR extracellular domain (GHRed) components of 1B7Stop (Human) with their respective bovine genes. The following sequences were gene synthesised:—

1) NheI-bovGHss-bovGH-NotI*
atatatgctagcccaccatgatggctgcaggcccccggacctccctgctc
ctggctttcgccctgctctgcctgccctggactcaggtggtgggcgccTT
CCCAGCCATGTCCTTGTCCGGCCTGTTTGCCAACGCTGTGCTCCGGGCTC
AGCACCTGCATCAGCTGGCTGCTGACACCTTCAAAGAGTTTGAGCGCACC
TACATCCCGGAGGGACAGAGATACTCCATCCAGAACACCCAGGTTGCCTT
CTGCTTCTCTGAAACCATCCCGGCCCCCACGGGCAAGAATGAGGCCCAGC
AGAAATCAGACTTGGAGCTGCTTCGCATCTCACTGCTCCTCATCCAGTCG
TGGCTTGGGCCCCTGCAGTTCCTCAGCAGAGTCTTCACCAACAGCTTGGT
GTTTGGCACCTCGGACCGTGTCTATGAGAAGCTGAAGGACCTGGAGGAAG
GCATCCTGGCCCTGATGCGGGAGCTGGAAGATGGCACCCCCCGGGCTGGG
CAGATCCTCAAGCAGACCTATGACAAATTTGACACAAACATGCGCAGTGA
CGACGCGCTGCTCAAGAACTACGGTCTGCTCTCCTGCTTCCGGAAGGACC
TGCATAAGACGGAGACGTACCTGAGGGTCATGAAGTGCCGCCGCTTCGGG
GAGGCCAGCTGTGCCTTCggcggccgcaattaattaatt
[gctagc = NheI site; [atg . . . gcc] = bovine GHss;
TTC . . . TTC = bovine GH; gcggccgc = NotI*
2) EcoRI-bovGHRab-HindIII
atatatgaattcTTTTCTGGGAGTGAAGCCACACCAGCTTTCCTTGTCAG
AGCATCTCAGAGTCTGCAGATACTATATCCAGTCCTAGAGACAAATTCTT
CTGGGAATCCTAAATTCACCAAGTGCCGTTCACCTGAACTGGAGACTTTC
TCATGTCACTGGACAGATGGGGCTAATCACAGTTTACAGAGCCCAGGATC
TGTACAGATGTTCTATATCAGAAGGGACATTCAAGAATGGAAAGAATGCC
CCGATTACGTCTCTGCTGGTGAAAACAGCTGTTACTTTAATTCGTCTTAT
ACCTCTGTGTGGACCCCCTACTGCATCAAGCTAACTAGCAATGGCGGTAT
TGTGGATCATAAGTGTTTCTCTGTTGAGGACATAGTACAACCAGATCCAC
CCGTTGGCCTCAACTGGACTCTACTGAACATCAGTTTGACAGAGATTCAT
GCCGACATCCTAGTGAAATGGGAACCACCACCCAATACAGATGTTAAGAT
GGGATGGATAATCCTGGAGTATGAACTGCACTATAAAGAACTAAATGAGA
CCCAGTGGAAAATGATGGACCCTTTAATGGTAACATCAGTTCCGATGTAC
TCGTTGAGACTGGATAAAGAGTATGAAGTGCGTGTGAGAACCAGACAACG
AAACACTGAAAAATATGGCAAGTTCAGTGAGGTGCTCCTGATAACATTTC
CTCAGATGAACCCAaagcttatatat
[gaattc = EcoRI site; TTT . . . CCA = bovine
GHRed; aagctt = HindIII]

These were digested with their respective end restriction enzymes (in bold) and sequentially ligated into the pGHSecTag.1B7Stop (Human) vector using the necessary restriction enzymes.

The new plasmid pGHSecTag.1B7Stop (Bovine) was verified by sequencing.

### Expression

The pGHSecTag.1B7Stop (Bovine) was transfected into Flp-In CHO cells and processed to produce stably expressing cells. The cells were then made into a suspension culture and protein expressed and secreted into the growth media.

### Purification

Antibodies against bovine GH or bovine GHRed were immobilised onto a purification column and this used to purify 1B7Stop(Bovine) from the media used to grow the Flp-In CHO cells stably transfected with pGHSecTag.1B7Stop(Bovine).

Purification of GH-GHR chimeras: Human GH and GH receptor were amplified by RT-PCR from human pituitary and liver respectively and cloned into the vector, pSecTag-V5/FRT/Hist-TOPO (Invitrogen, Paisley, UK) under the human GH secretion signal sequence. Four repeats of a Gly₄Ser linker were used to link the native C-terminus of Human GH to the native N-terminus of the Human GHR. Stable clones were made in CHO Flp-In cells (Invitrogen, Paisley, UK), adapted to protein free media and grown in suspension culture. Chimera expression was confirmed by an in-house GH ELISA. Affinity purification was performed using an anti-GH antibody column. Purity was determined by SDS-PAGE analysis followed by coomassie staining and western blotting using GH specific antibodies.

GHBP Binding: Displacement of ¹²⁵I-labeled GH binding to GHBP by unlabeled GH or chimera was studied by an immunoprecipitation method as previously described²⁶.

Transcription bioassays: These were performed as previously described in human 293 cells stably expressing the human GHR. The activity stimulated by GH or chimera is the fold induction stimulated by GH, i.e. corrected luciferase value in GH stimulated cells divided by corrected luciferase value in unstimulated cells.

Pharmacokinetic studies: Seven weeks old normal Sprague Dawley rats from Janvier (Le Genest Saint Isle, France) have been used for pharmacokinetic studies. Subcutaneous administration or intravenous administration (penile vein) and blood withdrawal (orbital sinus) were conducted under isoflurane anaesthesia. The rats (n=4-6/group) were injected iv or sc with rhGH or GH Chimera.

Growth studies: The growth studies used hypophysectomized rats and were performed on Sprague Dawley rats from Charles River laboratories (Larbresle, France). Rats were hypophysectomized under isoflurane anaesthesia at 4 weeks of age by the breeder and delivered 1 week after selection on body weight criteria for successful surgery. Animals were individually caged and allowed one other week of rest before entering the experimental phase. The injection solutions of excipient, rhGH and Chimera never exceed 2 ml/kg. The rats were weighed daily and depending on the administration protocol, received injections of the test substances for 10 days.

Characterisation of chimeras: Both denaturing, native gels and western blotting were used to analyse the chimera. Molecular weight was defined by gel filtration using a Superose G200 analytical column. Conformation of the chimera was examined using a panel of 16 conformationally sensitive anti-hGH receptor monoclonal antibodies. In the experiment, the chimera was immobilized directly to the microtiter plate or indirectly with capture antibodies, then detected by different monoclonal antibodies. These 16 mAbs were from different origins and were produced by immunizing the mice with recombinant nonglycosylated full length hGHR ECD produced in E. coli, or recombinant full length glycisylated hGHR ECD produced in baby Hamster kidney cells (BHK) or GHR purified from rat and rabbit liver. They have different binding epitopes, which cover the most parts of hGHR ECD and can be divided into 5 groups. These mAbs were all conformationally sensitive, as they can bind hGHBP in ELISA with high affinity, however do not bind the denatured (reduced) hGHBP in Western blot.

Statistics: The paired t-test was used with Bonferroni correction for multiple comparisons. For analysis of repeated measures ANOVA was used with Bonferroni correction as appropriate. Data are expressed as the mean±SEM, and a p<0.05 was considered to indicate statistically significant differences.

### Example 1

Purification of GH-GHR Chimera: Using a flexible Gly₄Ser linker with 4 repeats we fused native human GH to the A & B domains of the extracellular domain of the GH receptor. This 75 kDa chimera was expressed in CHO cells and purified using an anti-GH mAb affinity column to >95% purity (FIG. 2). The chimera appeared to purify as two bands with a difference in size of approximately 5 kDa.

### Example 2

Binding studies: The affinity of our chimeric molecule to GH receptor was tested in solution against recombinant GH binding protein with displacement of iodinated GH by unlabelled chimera. The 75 kDa chimera had a five-fold lower affinity than GH (Ka×10⁹ M⁻¹: 0.6±0.01 vs 3.1±0.03, respectively).

### Example 3

In vitro bioactivity of GH-GHR Chimeras in transcription bioassay (FIG. 3): The in vitro bioactivity of the chimera was tested using a GH-specific luciferase reporter assay. Essentially a human derived cell line was stably transfected with the human GH receptor and then transiently transfected with a luciferase signalling reporter. This assay detects physiological levels of GH. The chimera had only 10% of the bioactivity compared to GH, but 10-fold greater concentration of chimera than GH could still stimulate a maximal response in the bioassay.

### Example 4

Pharmacokinetic profile of GH-GHR chimeras (FIG. 4 and Table 1): Serum levels of the GH and chimera were measured as a function of time after a single subcutaneous (sc) or intravenous (iv) injection into normal rats. The chimera demonstrated both delayed absorption and delayed clearance and initial studies were performed over 6 hours (FIG. 4a) and subsequent studies over 8 days (FIG. 4b&c). After an iv bolus of the chimera, the clearance of the chimera was calculated to be 3.3±0.9 ml hrs⁻¹·kg⁻¹, and the volume of distribution was 46.0±3.3 ml·kg⁻¹. The calculated plasma half life was 21±2 hrs. Data are summarized in table 1.

### Example 5

Efficacy studies of GH-GHR chimeras in hypophysectomised rats (FIG. 5 and Table 2): To test biological activity, the chimera and GH were administered to hypophysectomised rats. Daily administration of GH induced continuous growth over 10 days with a mean±sem weight gain of 16.4±0.8%. The 75 kDa chimera was then compared to GH with either alternate day injections or two injections over 10 days at days 1 and 5. For all experiments equimolar doses of GH and chimera were used with the same total dose being given over the 10 day period irrespective of the injection protocol. The concentration of GH used was 220 μg/kg/day (1 nmol/100 g/rat/day) that equates to approximately 10 nmol over 10 days. The chimera promoted an increase in weight gain which was greater than GH when compared under the same injection protocol. GH appeared to only promote weight gain in the 24 hours post injection. In contrast the 75 kDa chimera produced continuous weight gain even when given as only two injections. The percentage weight gain over 10 days for two injections of the 75 kDa chimera was similar to that for daily injections of GH. A similar pattern of growth was seen in femur and tibia weight and length (Table 2). There was no difference in liver and kidney weight in GH and Chimera treated animals treated versus placebo treated animals, but the thymus showed a similar pattern in weight gain to that seen with whole body weight. The terminal bleed from all animals was analysed for IGF-I levels and measurement of GH and Chimera concentration (Table 2). IGF-I levels were significantly elevated after 75 kDa chimera in both treatment regimens (329±35 and 205±5 nM) and IGF-I levels were greater than those seen after daily injections of GH (92±30 nM). Levels of GH were undetectable in the terminal bleed after all injection regimens whereas 75 kDa chimera levels in the terminal bleed were 44±15 nM after alternate days injections and 23±5 nM after injections only every 5 days.

### Example 7

Characterisation of 75 kDa chimera: The chimera was screened by ELISA using a panel of 16 conformationally sensitive mAbs that cover epitopes in the extracellular domain of the GHR including the GH binding domain, the putative receptor dimerisation domain, and epitopes throughout the A & B domains of the GHR. All these mAbs bind the chimera with affinity comparable to their binding to GHBP from human serum (Table 3). These results indicate that the chimera has a similar conformation to native GHR. Coomassie staining and western blotting of SDS-PAGE gels (FIG. 2) showed the chimeric protein to separate as a consistent double band of approximately 75 kDa with an approximate 5 kDa difference between the two bands. Native PAGE gel analysis (FIG. 6) showed no evidence of aggregation but rather the chimera running as two distinct bands. The evidence for the existence of two forms in solution was confirmed by analytical gel filtration in which the chimeric protein separated as two distinct elution peaks, with the higher molecular weight form approximately twice the size of the lower molecular weight form. The chimera is glycosylated and therefore it was not possible to accurately size the molecule, but these results would be consistent with the chimera existing as at least a dimer in solution. Further analysis of the two distinct protein forms by SDS-PAGE under both reducing and non-reducing conditions showed that both forms consisted of the 75 kDa doublet (FIG. 6). Again these results are in keeping with the theory that a proportion of the chimera exists as a dimer in solution.

TABLE 1
Pharmacokinetic parameters in rats given a single
administration of the hGH and the chimera.

| | | Effective | Clearance | |
|---|---|---|---|---|
| Molecules | N | size (kDa) | (ml · hrs⁻¹ · kg⁻¹) | T_{1/2} (hrs) |
| hGH* | 6 | 22 | 496 ± 86 | 1.35 ± 0.2 |
| hGH-GHR Chimera | 6 | 75 | 3.3 ± 0.9 | 21 ± 2 |

TABLE 2
Results (mean ± sem) after 10 days treatment with GH or Chimera in hypophysectomised rats

| | | ×10 | ×5 | | | ×2 | | |
|---|---|---|---|---|---|---|---|---|
| | | (daily injections) | (injections every 2 days) | | | (injections every 5 days) | | |
| Variable at 10 days | Placebo | GH | GH | Chimera | t-test p | GH | Chimera | t-test p |
| Weight | 86.3 ± 1.6 | 103.3 ± 1.4 | 95.9 ± 0.8 | 102.2 ± 1.6 | <0.0001 | 88.4 ± 2.1 | 101.1 ± 0.7 | <0.0001 |
| Change in weight | 1.43 ± 0.96 | 16.4 ± 0.8 | 9.9 ± 0.5 | 17 ± 1.5 | 0.0003 | 4.5 ± 1.3 | 14.8 ± 0.9 | <0.0001 |
| from baseline | | | | | | | | |
| Femur Length | 0.00 ± 0.25 | 0.83 ± 0.26 | 0.99 ± 0.18 | 1.08 ± 0.07 | 0.667 | 0.44 ± 0.21 | 1.29 ± 0.22 | 0.0194 |
| Tibia weight (g) | 0.00 ± 0.02 | 0.03 ± 0.01 | 0.06 ± 0.02 | 0.05 ± 0.01 | 0.52 | 0.01 ± 0.01 | 0.07 ± 0.02 | 0.027 |
| Thymus weight (mg) | 0.00 ± 21 | 79 ± 20 | 43 ± 6 | 142 ± 22 | 0.0054 | 35 ± 12 | 120 ± 15 | 0.0132 |
| Liver weight (mg) | 0 ± 167 | 123 ± 170 | 362 ± 74 | 587 ± 206 | 0.056 | 402 ± 236 | 407 ± 116 | 0.073 |
| Kidney weight | | | | | | | | |
| IGF-I (ng · ml⁻¹) | 51 ± 12 | 92 ± 30 | 92 ± 30 | 329 ± 35 | 0.0005 | 55 ± 15 | 205 ± 5 | <0.0001 |
| GH or Chimera by | Nd | nd | nd | 44 ± 15 | 0.015 | nd | 23 ± 5 | 0.0015 |
| ELISA (nM) | | | | | | | | |

nd = Not Detectable

TABLE 3
Screening of the chimera with conformationally sensitiveanti-GHR mAbs

| | | | | GH binding | | rec. hGHBP | serum | |
|---|---|---|---|---|---|---|---|---|
| mAbs | Isotypes | antigen (GHBP) | domain(s) | site | groups | from E. coli | hGHBP | chimera |
| 2C8 | IgG1, k | from E. coli | A | yes | I | ++ | ++ | ++ |
| 3D2 | IgG1, k | from E. coli | A | yes | I | ++ | ++ | ++ |
| 5C6 | IgG2b, k | from E. coli | A | yes | I | ++ | ++ | ++ |
| 6C3 | IgG1, k | from E. coli | A | yes | I | ++ | ++ | ++ |
| 6F5 | IgG1, k | from E. coli | A | yes | I | ++ | ++ | ++ |
| B07b | IgG1, k | from BHK | A | yes | I | ++ | ++ | ++ |
| B24a | IgG1, k | from BHK | A | yes | I | ++ | ++ | ++ |
| 4B8 | IgG1, k | from E. coli | A&B | yes | II | ++ | ++ | ++ |
| 8B1 | IgG1, k | from E. coli | A&B | yes | II | ++ | ++ | ++ |
| 8F4 | IgG1, k | from E. coli | A&B | yes | II | ++ | ++ | ++ |
| 7A12 | IgG1, k | from E. coli | A&B | yes | II | ++ | ++ | ++ |
| 1H12 | IgG1, k | from E. coli | A | no | III | ++ | + | + |
| 263 | IgG1, k | from rat/rabbit | A | no | III | ++ | ++ | ++ |
| 3G4 | IgG1, k | from E. coli | hinge | no | VI | ++ | + | + |
| | | | region | | | | | |
| 2B3 | IgG1, k | from E. coli | B | no | VI | ++ | + | + |
| 9H12 | IgG1, k | from E. coli | B | no | VI | ++ | ++ | ++ |

1-44. (canceled)

45. A method to enhance the growth and/or metabolism of a non-human animal species comprising administering to said animal an effective amount of a chimeric fusion protein wherein said fusion protein comprises a first part comprising growth hormone, or a receptor binding domain thereof, optionally linked by a peptide linking molecule to a second part comprising the extracellular binding domain of growth hormone receptor.

46. The method according to claim 45 wherein said peptide linking molecule consists of 5-30 amino acid residues.

47. The method according to claim 45 wherein said chimeric fusion protein comprises an amino acid sequence as represented in FIG. 7b, or a variant amino acid sequence that varies from the sequence represented in FIG. 7b by addition, deletion or substitution of at least one amino acid residue and has the specific activity associated with growth hormone.

48. The method according to claim 45 wherein said chimeric fusion protein comprises an amino acid sequence as represented in FIG. 8b, or a variant amino acid sequence that varies from the sequence represented in FIG. 8b by addition, deletion or substitution of at least one amino acid residue and has the specific activity associated with growth hormone receptor.

49. The method according to claim 45 wherein said chimeric fusion protein is administered intravenously.

50. The method according to claim 45 wherein said chimeric fusion protein is administered subcutaneously.

51. The method according to claim 45 wherein said chimeric fusion protein is administered at two day intervals to said animal.

52. The method according to claim 45 wherein said fusion protein is administered at monthly intervals to said animal.

53. The method according to claim 45 wherein said fusion protein is administered at 2 weekly intervals to said animal.

54. The method according to claim 45 wherein said fusion protein is administered at weekly intervals to said animal.

55. The method according to claim 45 wherein said fusion protein is administered at 2 daily intervals to said animal.

56. The method according to claim 45 wherein said animal is selected from the group consisting of: cow, sheep, pig, horse, deer, boar, fowl or fish.

57. The method according to claim 56 wherein said animal is a cow.

58. A nucleic acid molecule that encodes a polypeptide as represented by the amino acid sequence in FIG. 9b.

59. A nucleic acid molecule that encodes a polypeptide as represented by the amino acid sequence in FIG. 10b.

60. A polypeptide comprising an amino acid sequence as represented in FIG. 9b.

61. A polypeptide comprising an amino acid sequence as represented in FIG. 10b.

62. An expression vector comprising a nucleic acid molecule according to claim 58 or 59.

63. A cell transfected with a vector according to claim 62.

64. A homodimer comprising first and second polypeptides wherein said first and second polypeptides comprise: a first part comprising growth hormone, or a receptor binding domain thereof, optionally linked by a peptide linking molecule to a second part comprising the extracellular binding domain of growth hormone receptor.

65. The homodimer according to claim 64 wherein said growth hormone and the extracellular binding domain of growth hormone receptor are bovine.

66. The homodimer according to claim 65 wherein said homodimer comprises a polypeptide comprising or consisting of an amino acid sequence as represented in FIGS. 9b, 10b, 11b or 12b.


SEQUENCE LISTING (15 sequences)

SEQ ID NO: 1 - DNA, 674 bp, Bos taurus
attaaagcttatgatggctgcaggcccccggacctccctgctcctggctttcgccctgctctgcctgccctggactcaggtggtgggcgccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggagctggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgtgccttctaggaattcacta

SEQ ID NO: 2 - protein, 190 aa, Bos taurus
FPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAF

SEQ ID NO: 3 - DNA, 2014 bp, Bos taurus
tctctctcccgaggctcggctcgcaggtcctacaggtatggatctctggcagctgctgttgaccttggcagtggcaggctccagtgatgctttttctgggagtgaagccacaccagctttccttgtcagagcatctcagagtctgcagatactatatccagtcctagagacaaattcttctgggaatcctaaattcaccaagtgccgttcacctgaactggagactttctcatgtcactggacagatggggctaatcacagtttacagagcccaggatctgtacagatgttctatatcagaagggacattcaagaatggaaagaatgccccgattacgtctctgctggtgaaaacagctgttactttaattcgtcttatacctctgtgtggaccccctactgcatcaagctaactagcaatggcggtattgtggatcataagtgtttctctgttgaggacatagtacaaccagatccacccgttggcctcaactggactctactgaacatcagtttgacagagattcatgccgacatcctagtgaaatgggaaccaccacccaatacagatgttaagatgggatggataatcctggagtatgaactgcactataaagaactaaatgagacccagtggaaaatgatggaccctttaatggtaacatcagttccgatgtactcgttgagactggataaagagtatgaagtgcgtgtgagaaccagacaacgaaacactgaaaaatatggcaagttcagtgaggtgctcctgataacatttcctcagatgaacccatctgcatgtgaagaagatttccagtttccatggttcttaattattatctttggaatacttgggctagcagtgacattatatttactcatattttctaaacagcaaaggattaagatgctgattctacccccagttccagttccaaagattaaaggaattgatccagatctcctcaaggaaggaaaattagaagaggtgaatacaatcttagccattcatgacaactataaacacgaattctacaatgatgactcttgggttgaatttattgaactagatattgatgaccctgatgaaaagactgaagggtcagacacagacagacttctgagcaatgaccatgaaaaatcactcaatatctttggggcaaaggatgacgactctgggcgtaccagctgctatgaacctgacattctggaggctgatttccatgtcagtgacatgtgcgatggtacctcagaggttgctcagccacaaaggttaaaaggggaagcagatatctcatgccttgatcagaagaatcaaaataattcaccttctaatgatgctgcccctgctagccagcagcccagtgttatcctagtagaggaaaacaaaccaagaccacttctcattggtggaactgagtcaactcatcaagctgtccatacacagctcagcaatccaagttcattggcaaacattgatttttatgcccaggtaagcgacattacaccagcaggaaatgtggtcctttccccaggccaaaagaataagactgggaacccccagtgtgacacgcacccagaagtggtcacaccctgccaagctaacttcatcgtggacaacgcttacttctgcgaggtagacgccaaaaagtacattgccctggcccctcatgtcgaggctgaatcacacatagagccaagctttaaccaggaagacatttacatcaccacagaaagccttaccactacagctgggaggtcggggacagcagaacatgttccaagttctgagatacctgtcccagattatacctccattcatatagtacagtctccacagggcctcgtactcaatgcgactgccctgcccttgcctgacaaagagtttctctcatcatgtggctatgtgagcacagaccaactgaacaaaatcatgccatagcttttctttgatttcctatgagctacccattgaatggcacagggttggctggggcatgaatgcttaaaccaa

SEQ ID NO: 4 - protein, 634 aa, Bos taurus
MDLWQLLLTLAVAGSSDAFSGSEATPAFLVRASQSLQILYPVLETNSSGNPKFTKCRSPELETFSCHWTDGANHSLQSPGSVQMFYIRRDIQEWKECPDYVSAGENSCYFNSSYTSVWTPYCIKLTSNGGIVDHKCFSVEDIVQPDPPVGLNWTLLNISLTEIHADILVKWEPPPNTDVKMGWIILEYELHYKELNETQWKMMDPLMVTSVPMYSLRLDKEYEVRVRTRQRNTEKYGKFSEVLLITFPQMNPSACEEDFQFPWFLIIIFGILGLAVTLYLLIFSKQQRIKMLILPPVPVPKIKGIDPDLLKEGKLEEVNTILAIHDNYKHEFYNDDSWVEFIELDIDDPDEKTEGSDTDRLLSNDHEKSLNIFGAKDDDSGRTSCYEPDILEADFHVSDMCDGTSEVAQPQRLKGEADISCLDQKNQNNSPSNDAAPASQQPSVILVEENKPRPLLIGGTESTHQAVHTQLSNPSSLANIDFYAQVSDITPAGNVVLSPGQKNKTGNPQCDTHPEVVTPCQANFIVDNAYFCEVDAKKYIALAPHVEAESHIEPSFNQEDIYITTESLTTTAGRSGTAEHVPSSEIPVPDYTSIHIVQSPQGLVLNATALPLPDKEFLSSCGYVSTDQLNKIMP

SEQ ID NO: 5 - DNA, 689 bp, Artificial
  bovine growth hormone used in cloning construction of chimera
atatatgctagcccaccatgatggctgcaggcccccggacctccctgctcctggctttcgccctgctctgcctgccctggactcaggtggtgggcgccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggagctggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgtgccttcggcggccgcaattaattaatt

SEQ ID NO: 6 - DNA, 726 bp, Artificial
  bovine growth hormone receptor used in construction of chimera
atatatgaattcttttctgggagtgaagccacaccagctttccttgtcagagcatctcagagtctgcagatactatatccagtcctagagacaaattcttctgggaatcctaaattcaccaagtgccgttcacctgaactggagactttctcatgtcactggacagatggggctaatcacagtttacagagcccaggatctgtacagatgttctatatcagaagggacattcaagaatggaaagaatgccccgattacgtctctgctggtgaaaacagctgttactttaattcgtcttatacctctgtgtggaccccctactgcatcaagctaactagcaatggcggtattgtggatcataagtgtttctctgttgaggacatagtacaaccagatccacccgttggcctcaactggactctactgaacatcagtttgacagagattcatgccgacatcctagtgaaatgggaaccaccacccaatacagatgttaagatgggatggataatcctggagtatgaactgcactataaagaactaaatgagacccagtggaaaatgatggaccctttaatggtaacatcagttccgatgtactcgttgagactggataaagagtatgaagtgcgtgtgagaaccagacaacgaaacactgaaaaatatggcaagttcagtgaggtgctcctgataacatttcctcagatgaacccaaagcttatatat

SEQ ID NO: 7 - DNA, 1428 bp, Artificial
  bovine growth hormone fusion protein
atgatggctgcaggcccccggacctccctgctcctggctttcgccctgctctgcctgccctggactcaggtggtgggcgccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggagctggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgtgccttcggcggccgcggtggcggaggtagtggtggcggaggtagcggtggcggaggttctggtggcggaggttccgaattcttttctgggagtgaagccacaccagctttccttgtcagagcatctcagagtctgcagatactatatccagtcctagagacaaattcttctgggaatcctaaattcaccaagtgccgttcacctgaactggagactttctcatgtcactggacagatggggctaatcacagtttacagagcccaggatctgtacagatgttctatatcagaagggacattcaagaatggaaagaatgccccgattacgtctctgctggtgaaaacagctgttactttaattcgtcttatacctctgtgtggaccccctactgcatcaagctaactagcaatggcggtattgtggatcataagtgtttctctgttgaggacatagtacaaccagatccacccgttggcctcaactggactctactgaacatcagtttgacagagattcatgccgacatcctagtgaaatgggaaccaccacccaatacagatgttaagatgggatggataatcctggagtatgaactgcactataaagaactaaatgagacccagtggaaaatgatggaccctttaatggtaacatcagttccgatgtactcgttgagactggataaagagtatgaagtgcgtgtgagaaccagacaacgaaacactgaaaaatatggcaagttcagtgaggtgctcctgataacatttcctcagatgaaccca

SEQ ID NO: 8 - protein, 453 aa, Artificial
  bovine growth hormone chimera
FPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAFGGRGGGGSGGGGSGGGGSGGGGSEFFSGSEATPAFLVRASQSLQILYPVLETNSSGNPKFTKCRSPELETFSCHWTDGANHSLQSPGSVQMFYIRRDIQEWKECPDYVSAGENSCYFNSSYTSVWTPYCIKLTSNGGIVDHKCFSVEDIVQPDPPVGLNWTLLNISLTEIHADILVKWEPPPNTDVKMGWIILEYELHYKELNETQWKMMDPLMVTSVPMYSLRLDKEYEVRVRTRQRNTEKYGKFSEVLLITFPQMNPKLFE

SEQ ID NO: 9 - DNA, 1428 bp, Artificial; identical to SEQ ID NO: 7
  bovine growth hormone chimera

SEQ ID NO: 10 - protein, 449 aa, Artificial
  bovine growth hormone chimera
FPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAFGGRGGGGSGGGGSGGGGSGGGGSEFFSGSEATPAFLVRASQSLQILYPVLETNSSGNPKFTKCRSPELETFSCHWTDGANHSLQSPGSVQMFYIRRDIQEWKECPDYVSAGENSCYFNSSYTSVWTPYCIKLTSNGGIVDHKCFSVEDIVQPDPPVGLNWTLLNISLTEIHADILVKWEPPPNTDVKMGWIILEYELHYKELNETQWKMMDPLMVTSVPMYSLRLDKEYEVRVRTRQRNTEKYGKFSEVLLITFPQMNP

SEQ ID NO: 11 - DNA, 1428 bp, Artificial
  bovine growth hormone chimera
atgatggctgcaggcccccggacctccctgctcctggctttcgccctgctctgcctgccctggactcaggtggtgggcgccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggagctggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgtgccttcggtggcggaggtagtggtggcggaggtagcggtggcggaggttctggtggcggaggttccggtggcggaggtagtttttctgggagtgaagccacaccagctttccttgtcagagcatctcagagtctgcagatactatatccagtcctagagacaaattcttctgggaatcctaaattcaccaagtgccgttcacctgaactggagactttctcatgtcactggacagatggggctaatcacagtttacagagcccaggatctgtacagatgttctatatcagaagggacattcaagaatggaaagaatgccccgattacgtctctgctggtgaaaacagctgttactttaattcgtcttatacctctgtgtggaccccctactgcatcaagctaactagcaatggcggtattgtggatcataagtgtttctctgttgaggacatagtacaaccagatccacccgttggcctcaactggactctactgaacatcagtttgacagagattcatgccgacatcctagtgaaatgggaaccaccacccaatacagatgttaagatgggatggataatcctggagtatgaactgcactataaagaactaaatgagacccagtggaaaatgatggaccctttaatggtaacatcagttccgatgtactcgttgagactggataaagagtatgaagtgcgtgtgagaaccagacaacgaaacactgaaaaatatggcaagttcagtgaggtgctcctgataacatttcctcagatgaaccca

SEQ ID NO: 12 - protein, 449 aa, Artificial
  bovine growth hormone chimera
FPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAFGGGGSGGGGSGGGGSGGGGSGGGGSFSGSEATPAFLVRASQSLQILYPVLETNSSGNPKFTKCRSPELETFSCHWTDGANHSLQSPGSVQMFYIRRDIQEWKECPDYVSAGENSCYFNSSYTSVWTPYCIKLTSNGGIVDHKCFSVEDIVQPDPPVGLNWTLLNISLTEIHADILVKWEPPPNTDVKMGWIILEYELHYKELNETQWKMMDPLMVTSVPMYSLRLDKEYEVRVRTRQRNTEKYGKFSEVLLITFPQMNP

SEQ ID NO: 13 - DNA, 1353 bp, Artificial
  bovine growth hormone chimera
atgatggctgcaggcccccggacctccctgctcctggctttcgccctgctctgcctgccctggactcaggtggtgggcgccttcccagccatgtccttgtccggcctgtttgccaacgctgtgctccgggctcagcacctgcatcagctggctgctgacaccttcaaagagtttgagcgcacctacatcccggagggacagagatactccatccagaacacccaggttgccttctgcttctctgaaaccatcccggcccccacgggcaagaatgaggcccagcagaaatcagacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggagctggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgtgccttcttttctgggagtgaagccacaccagctttccttgtcagagcatctcagagtctgcagatactatatccagtcctagagacaaattcttctgggaatcctaaattcaccaagtgccgttcacctgaactggagactttctcatgtcactggacagatggggctaatcacagtttacagagcccaggatctgtacagatgttctatatcagaagggacattcaagaatggaaagaatgccccgattacgtctctgctggtgaaaacagctgttactttaattcgtcttatacctctgtgtggaccccctactgcatcaagctaactagcaatggcggtattgtggatcataagtgtttctctgttgaggacatagtacaaccagatccacccgttggcctcaactggactctactgaacatcagtttgacagagattcatgccgacatcctagtgaaatgggaaccaccacccaatacagatgttaagatgggatggataatcctggagtatgaactgcactataaagaactaaatgagacccagtggaaaatgatggaccctttaatggtaacatcagttccgatgtactcgttgagactggataaagagtatgaagtgcgtgtgagaaccagacaacgaaacactgaaaaatatggcaagttcagtgaggtgctcctgataacatttcctcagatgaaccca

SEQ ID NO: 14 - protein, 424 aa, Artificial
  bovine growth hormone chimera
FPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAFFSGSEATPAFLVRASQSLQILYPVLETNSSGNPKFTKCRSPELETFSCHWTDGANHSLQSPGSVQMFYIRRDIQEWKECPDYVSAGENSCYFNSSYTSVWTPYCIKLTSNGGIVDHKCFSVEDIVQPDPPVGLNWTLLNISLTEIHADILVKWEPPPNTDVKMGWIILEYELHYKELNETQWKMMDPLMVTSVPMYSLRLDKEYEVRVRTRQRNTEKYGKFSEVLLITFPQMNP

SEQ ID NO: 15 - protein, 5 aa, Artificial
  peptide linker to link growth hormone and growth hormone receptor polypeptides
GGGGS